(19) United States
(12) Patent Application Publication
(10) Pub. No.: US 2010/0186104 A1
(43) Pub. Date: Jul. 22, 2010

(54) Heat-Tolerant Cotton Plant Containing Multiple Transgenes

(75) Inventors: Sheikh Riazuddin, Lahore (PK); Tayyab Hussain, Lahore (PK)

Correspondence Address: SARFARAZ K. NIAZI, 20 RIVERSIDE DRIVE, DEERFIELD, IL, 60015, US

(73) Assignee: National Center of Excellence in Molecular Biology, Punjab University, Lahore (PK)

(21) Appl. No.: 12/357,257

(22) Filed: Jan. 21, 2009

(51) Int. Cl. A01H 1/00; A01H 5/00

(52) U.S. Cl. 800/265; 800/302

(57) ABSTRACT

The present invention provides methods to genetically transform cotton plant, Gossypium hirsutum, a heat-tolerant cotton variety to impart resistance to insect damage and to make stable hybrid variety for commercial use.

Cotton is a worldwide cash crop and is also used for cottonseed oil production; however, its fiber and oil yield depend greatly upon its photosynthetic ability and resistance to insect damage. Cotton is susceptible to attack and damage by more than 15 different insects that can destroy a substantial portion of the crop. These losses are currently managed, although inefficiently, by the use of chemical insecticides, which contaminate crops as well as add to the cost of crop substantially. Conventional breeding has proved useful in breeding genetic resistance to insects; however, no cotton variety exhibiting complete resistance to bollworms has been placed in commerce in many parts of the world including one of the largest cotton producing country, Pakistan.

The introduction of genes encoding insecticidal proteins from Bacillus thuringiensis (Bt) into plants to breed resistance against American bollworm, a major pest, has been successful and holds promise in reducing dependence on chemical pesticides. Several commercial varieties based on Bt genes have been produced but it is generally accepted that insect may become resistant to Bt toxin after prolonged and repeated exposures. To obviate this resistance risk, several strategies have been proposed to delay or avoid the process of resistance build up by insect against Bt protein. These strategies include gene pyramiding, crop rotation, high dose and spatial or temporal refugia.

Gene pyramiding is the simultaneous transfer of more than one gene into transgenic plants. The present invention discloses methods to transform cotton plant with multiple genes (CryIAc, Cry2A,) to achieve insect resistance improve yield and modify other biochemical characteristics of the cotton plants.

Bacillus thuringiensis (Bt) is a Gram-positive bacterium widely used as a source of genes for genetic transformation of cotton. Its biological activity mainly resides in its parasporal protein inclusion body or crystal (Aronson et al 1991, Hofte and Whiteley 1989). Bt produces several insecticidal crystalline proteins as the sporolation. These cry proteins include alpha (α), beta β, gamma (γ) and delta (Δ) endotoxins. Delta endotoxins are predominately synthesized as long inactive protoxins that are activated by proteolysis in the insect gut. Most strains are active against Lepidopteran larvae but some are toxic against dipteral, coleopteran species.

Perlak et al (1991) used two approaches to increase the level of Cry1A (b) and Cry1A (c) insect control proteins in genetically modified plants. First, DNA sequences predicted to inhibit efficient plant gene expression at both the translational and mRNA level were selectively removed throughout the coding sequences by site-directed mutagenesis to partially modify the gene (PM Gene) without changing the amino acid sequences. The second approach was creating fully modified synthetic gene (FM Gene). FM Gene encodes proteins nearly identical in amino acid sequences to the wild-type (WT) Gene. Among the most highly expressing transformed plants for each gene the plants with partially modified Cry 1A(b) gene had a 10-fold higher level of insect control protein and plant with FM Gene had 100-fold higher level of Cry1A (b) protein as compared to the WT gene.

In the present invention, we have created and used fully modified gene with higher GC contents to make higher transcription and translation in cotton plants.

Introduction of foreign genes in elite genotypes is limited by the genotype-specific nature of the gene transfer in cotton. Coker genotypes, which are emendable for regeneration in vitro by somatic embryogenesis, are widely used in genetic transformation. However, alternate procedure to transform non-coker genotypes has been reported. Kumar et al (1998) attempted to transfer the regenerative competence from Coker varieties to recalcitrant elite cultivars and developed a Coker 310 FR line, which could be used for genetic transformation. The transgene from the Coker 310 FR can then be transferred to elite genotypes by conventional breeding. However, this transference could also lead to introgression of undesirable characteristics from Coker 310FR. Thus transformation of elite genotype is desirable. We have developed SIDE method for introduction of transgene in various cotton varieties.

Heterosis is a universal phenomenon in living nature. Therefore it was speculated that exploitation of heterosis might be a quick way to incorporate resistance provided by Bt gene with other desired agronomic characters of the heat tolerant cotton varieties. The dominant inheritance of Bt gene provides possibilities for developing insect-resistant hybrids. Though genetic background influences the expression of Bt gene (Adamczyk and Sumerford 2001), hybrids can be obtained by selection of parents and combining ability tests. The method described in this invention has resulted in the transformation of elite cotton varieties with foreign genes.

FIG. 1 is a graphical representation of the sequence alignment for the DNA, which is flanking the inserted sequences and the inserted sequences themselves, including the cry1Ac gene in cotton event CEMB02-451. The inserted DNA is depicted as a single strand with 5′ from the left through 3′ to the right.

Individual DNA sequences identified herein are labeled in FIG. 1 as number 1-7) as follows:

Reference number 1 is the DNA sequence for the Cry1Ac sequence and its border sequence (SEQ ID NO: 1).

Reference number 2 is the DNA sequence for the Cry2A Sequence and its border sequence (SEQ ID NO: 2).

Reference number 3 is the sequence of forward Cry1Ac primer to amplify a 565 bp fragment (SEQ ID NO: 3).

Reference number 4 is the sequence of reverse Cry1Ac primer to amplify a 565 bp fragment (SEQ ID NO: 4).

Reference number 5 is the sequence of forward Cry2A primer to amplify a 600 bp fragment (SEQ ID NO: 5).

Reference number 6 is the sequence of Reverse Cry2A primer to amplify 600 bp fragment (SEQ ID NO: 6).

Reference number 7 is the sequence of cotton event CEMB02-451 (SEQ ID NO: 7).

SEQ ID NO: 8 is the sequence of forward primer of event CEMB02-451 to amplify 451 bp fragment.

SEQ ID NO: 9 is the sequence of Reverse primer of event CEMB02-451 to amplify 451 bp fragment.

SEQ IDs 2, 3, 5, 6, 8 and 9 were used as diagnostic sequence for CEMB cotton. A junction sequence herein spans the point at which DNA inserted into the genome is linked to DNA from the cotton native genome flanking the insertion point, the identification or detection of one or the other junction sequences in a plant's genetic material being sufficient to be diagnostic for the event. Included are the DNA sequences that span the insertions in cotton event CEMB02-451 and similar lengths of flanking DNA. Examples of such diagnostic sequences are SEQ IDs 8 and 9. Nucleic acid amplification of genomic DNA from the event, using the primers provided herein or designed by one of ordinary skill in the art, produces an amplicon comprising such diagnostic DNA sequences. In addition, detection of the binding of oligonucleotides, which bind specifically to the diagnostic sequences described herein is also diagnostic for the event.

### SUMMARY OF INVENTION

The invention is related to the development of new cotton lines containing the Bt genes to overcome the inherent deficiencies in the prior art by providing novel processes that will allow genetic transformation of elite varieties of cotton without crossing with unconventional cotton varieties such as the Coker-type. The present invention innovatively allows direct transformation of cotton varieties without crossing with any other variety obviating genetic contamination.

The present invention discloses processes for producing stably transformed elite cotton cultivars from freshly isolated or mature embryos. The invention also discloses methods for the improved introduction of the transgene by a novel method, named as SIDE (Sonication Induced DNA Entry) and regeneration within a short period of time resulting in a fertile insect-resistant plant. The transformation frequency by using this method is considerably higher than reported in any prior art. This invention also discloses methods to manipulate genes found in microbes isolated from arid and semi arid environments for increased translation and transcription in heat tolerant cotton varieties.

In view of the foregoing, the first aspect of the present invention is a method of making recombinant cotton plants that have reduced variability of transcription and translation and increased level of transcription and translation of microbial gene.

The second aspect of the present invention is a nucleotide sequence. Sequences from 1 to 36 were blasted against invented plant Cry genes sequence by using Clustalw and Bio edit as well as NCBI blast software. The results clearly indicate that there is no discernible homology between the invented Cry genes and other reported sequences.

The third aspect of the present invention is a DNA construction, inserted in plant transformation vector and used above.

The fourth aspect of the present invention is a plant containing DNA constructs as given above.

The fifth aspect of the present invention is a recombinant cotton plant containing genetic material from an inbred cotton line and cross with inbred line in Pakistan to develop hybrid containing a defined sequence of nucleotides, synthesized from the information of DNA isolated from arid and semi arid regional microbes, as above.

The main principal of the invention was to process arid and semi arid regional bacteria, isolate sequence and modify them for optimized expression in plant and to transform cotton varieties CIM-497, CIM-482, CIM-446, MNH-93, CIM-443 and others with insect-resistant gene by using the SIDE method. Plants were regenerated, screened for the presence and functioning of introduced genes and field trials were conducted to assess the performance of the newly developed cotton variety under severe attack of Lepidopteran, the sucking insects.

### DETAILS OF INVENTION

Tissue Culture of Heat Tolerant G. hirsutum:

Seeds of the variety Gossypium hirsutum were delinted and sterilized by treating with a solution containing a few drops of Tween 20; these were later treated with 50% sodium hypochlorite solution for 20 minutes followed by 5 washings with autoclaved distilled water, and the final washing for 1 hr. The seeds were the kept in dark for germination at 30° C. wherein embryo were isolated for these seeds under aseptic conditions. Tissue culture and regeneration media were based on the formulation as described by Murashige and Skoog (1962).

Agrobacterium tumefaicens Mediated Transformation:

Agrobacterium strain EHA105 with plasmid pCMAC was used for the comparison of transit expression. From glycerol stock stored at −70° C., EHA105 was streaked on YEP (Chilton et al. 1974) medium containing Kanamycin (50 μgm/ml). The control strain of EHA 105 (without pCMAC) was also streaked on the same medium for 48 hours on 25±2° C. After 48 hours, a single colony was picked to inoculate 10 ml of YEP liquid medium containing 50 μg/ml of kanamycin in 50 ml culture tube. The control YEP liquid medium was also used which contained autoclaved toothpick. Culture tubes were placed in rotary shaker at speed of 200 rpm overnight and the next morning 100 ul of culture were spread on the YEP plant containing 50 μl of kanamycin. Plates were incubated at 20±2° C. for 48 hours. Rest of the liquid culture and streaked plates were placed at 4° C. to be stored for one week. After 48 hours, bacterial lawn was scraped from one culture plate and dissolved in 100 ml of MS liquid medium. Mature cotton embryos were isolated and incubated with Agrobacterium strain EHA 105 on rotary shaker, at the speed of 200 rpm for half an hour. Then embryos were dried on filter paper and transferred on MS medium and co-cultivated at 25±2° C. for 48 hours.

Transformation of Cotton:

Conditions were also developed to transform cotton varieties mentioned above with marker as well as genes mentioned above by the SIDE method. The plants were generated and analyzed for the transcription and translation of Cry1Ac, Cry2A.

Molecular and Entomological Analysis:

Various tests were preformed to find out the integration and expression of the respective genes in Cotton including Dot blot, PCR amplification, southern hybridization and entomological analysis.

Field Trial of Transgenic Lines:

The first generation of plants containing the respective photosynthetic and insect resistance genes were grown in experimental fields of the Center for Excellence in Molecular Biology (Lahore Pakistan) in compliance with the recommended biosafety guidelines. The respective transgenic plants were assessed for various respective parameters and the photosynthetic performance based on the leaf area and number of leaves and resistance to insects, while the non-transformed plants served as a control. The damage was determined at vegetative as well as at maturing stage.

Sonicated Induced DNA Entry (SIDE):

Sonication, incubation and co-cullivation of isolated embryos with Agrobacterium were performed as described earlier. The seeds of the heat tolerant cotton variety CIM-482 were sterilized as described above. After sterilization, the seeds were soaked in autoclaved distilled water for one hour. After one hour, excess water was removed and the seeds were kept in the dark at 30° C. overnight for germination. The next morning, testa of the seeds was removed carefully with a forceps and the cotyledonary leaves were excised with surgical blades. The mature cotton embryos were isolated from the germinating seeds. During this isolation process, the isolated embryos were kept on moist filter paper to prevent them from drying.

The mature cotton embryos (approximately 500) after isolation from the seeds were shifted to 50 ml polystyrene centrifuge tube containing 10 ml MS broth. An electronic timer controlled the number of pulses on sonicator as 3,6,9,12,15 and 18 pulses. The tip of sonicator was washed with 70% ethanol. Then sonicator was used at different number of pulses. After sonication treatment, embryos were immediately shifted to the Agrobacterium inoculums suspension for treatment with bacterial culture for 1 hour on a rotary shaker at very slow speed. A total of approximately 8,000 embryos were used in the transformation experiments.

Plants were subcultured on selection medium containing 10 ug/ml of kanamycin for selection of transformed plants and cefotaxime 250 μg/ml to kill Agrobacterium for 6-8 weeks before transferring to selection free medium for root formation. Control plants were also carried along with the co-cultivated plants on both selection medium (negative control) and selection free medium (positive control).

Polymerase Chain Reaction

PCR conditions were: 95° C. for 3 minutes; 94° C. for 45 seconds; 56° C. for 45 seconds and 72° C. for 1 minute (35 cycles). A final cycle was carried out at 72° C. for 7 minutes.

PCR for Cry1Ac

The DNA from the leaves of transgenic plants was analyzed by PCR. The DNA extraction was done using Plant Phytopure DNA extraction kits (Amersham). PCR analyses were carried out by using specific primers for amplification of a 565 bp fragment from the Cry1Ac gene.

The DNA extracted from untransformed plant was used as control while plasmid DNA (10 ng) was used as positive control. PCR reaction was carried out in 25 μl reaction volume using genomic DNA (as template) 100 ng, forward and reverse primers 50 pM each, dNTPs 200 mM Tris-HCl pH 9.0) and Taq polymerase 1 unit. The PCR conditions were: 95° C. for 5 minutes; 94° C. for 45 seconds; 52° C. for 45 seconds and 72° C. for 45 seconds (35 cycles). A final cycle was carried out at 72° C. for 7 minutes.

PCR Cry2A

The DNA from the leaves of transgenic plants was analyzed by PCR. The DNA extraction was done using Plant Phytopure DNA extraction kits (Amersham). PCR analyses were carried out by using specific primers for amplification of a 600 bp fragment from the Cry2A gene.

DNA extracted from untransformed plant was used as control while plasmid DNA (10 ng) was used as positive control. PCR reaction was carried out in 25 μl reaction volume using genomic DNA (as template) 100 ng, forward and reverse primers 50 pM each, dNTPs 200 mM Tris-HCl pH 9.0) and Taq polymerase 1 unit. The PCR conditions were 94° C. for 45 seconds, 60° C. for 45 seconds and 72° C. for 45 seconds (35 cycles). A final cycle was carried out at 72° C. for 7 minutes.

Dot Blot

Five μg of plant DNA was diluted in 1.0 μl of water denatured in boiling water bath and quickly chilled on ice. The DNA was spotted on nitrocellulose membrane. DNA fixation was done by incubating at 130° C. for 30 minutes. DNA from untransformed control plants was also isolated and spotted in the same way. 10 ng of plasmid DNA was used as positive control. DIG labeling kit (Boehringer Mannheim) was used and probe was prepared according to manufacture's instructions.

Standard capillary transfer (Southern, 1975) was used to blot DNA onto nylon (Hi-bond) membrane. The DNA was blotted from gel to membrane using 20×SSC (1.5 M NaCl and 0.1 M sodium citrate) for 16 hours. Transfer membranes were baked at 130° C. for 30 minutes. Prehybridization, hybridization and detection of the labeling signals was done according to Genius manual.

Publications

Adamczyk, J. J. and Sumerford, D. V. (2001). Genetic background influences the expression of Bt gene J. Insect. Sci. 1, 13 16

Ali, R. M., Husnain, T., Hussain, S. S., Mahmood, N., and Riazuddin. S. (2004). Multiple Shoot regeneration response of recalcitrant cotton (Gossypium hirsutum) cultivar CIM-443.PJBS 7(8). 1371-1375.

Aronson A. I., E. S., McCaughey, W and Johnson, D. (1991). The solubility of inclusion proteins from Bacillus thuringiensis is dependent upon protoxin composition and is a factor in toxicity to insects. Applied and Environmental Microbiology 57, 981-986

Chilton, M. D., Currier, T. C., Frand, S. K., Bendich, A. J., Gordon, M. P., Nester E. W., (1974). Agrobacterium tumefaciens DNA and PS8 bacteriophage DNA not detected in Crown Gall Tumors. Proc. Natl. Acad. Sci. USA. 71:3672-3676

Hofte, H and Whiteley H. R (1989). Insecticidal crystal proteins of Bacillus thuringiensis Microb Rev. 53 242-255.

Katageri, S. I., Vamadevaiah. M. H., Udikeri. S. S., Khadi and Polumetla A. Kumar (2007). Genetic transformation of an elite Indian genotype of cotton (Gossypium hirsutum L.) for insect resistance Current Science, Vol. 93, NO. 12.

Kumar S., Sharma P and Pental, D (1998). A genetic approach to in vivo regeneration of non regenerating cotton cultivars Plant Cell Report 59-63.

Murashige, T., and Skoog, F. (1962). A revised medium for rapid growth and bioassays with tobacco tissue cultivars. Plant Physiol. 150: 473-497.

Perlak, F. J., Fuchs, R. L., Dean, D. A., McPherson, S. L., Fischhoff, D. A. (1991). Modification of the coding sequence enhances plant expression of insect cotton protein genes. Proc. Natl Acad. Sci. USA, 88, 3324-3328.

Perlak, F. J., Deaton, R. W., Armstrong, T. A., Fuchs, R. L., Sims, S. R., Greenplate, J. T., Fischhoff, D. A. (1990) Insect resistant cotton plants. Bio/Technol. 8, 939-943.

SEQ ID NO: 1

GCACTTTCGTTCTTGACGGACAGAGTTCGCCTATGGAACCTCTTCTAACT
TGCCATCCGCTGTTTACAGAAAGAGCGGAACCGTTGATTCCTTGGACGAA
ATCCCACCACAGAACAACAATGTGCCACCCAGGCAAGGATTCTCCCACAG
GTTGAGCCACGTGTCCATGTTCCGTTCCGGATTCAGCAACAGTTCCGTGA
GCATCATCAGAGCTCCTATGTTCTCTTGGATACACCGTAGTGCTGACTTC
AACAACATCATCGCATCCGATAGTATTACTCAAATCCCTGCAGTGAAGGG
AAACTTTCTCTTCAACGGTTCTGTCATTTCAGGACCAGGATTCACTGGTG
GAGACCTCGTTAGACTCAACAGCAGTGGAAATAACATTCAGAATAGATGG
TATATTGAAGTTCCAATTCACTTCCCATCCACATCTACCAGATATAGAGT
TCGTGTGAGGTATGCTTCTGTGACCCCTATTCACCTCAACGTTAATTGGG
GTAATTCATCCATCTTCTCCAATACAGTTCCAGCTACAGCTACCTCCTTG
GATAATCTCCAATCCAGCGATTTCGGTTACTTTGAAAGTGCCAATGCTTT
TACATCTTCACTTCGGTAACCTTCAGTACGAATCGCTGGATGGAGATATC
CAAGGAGGTAGCTGTAGCTCGGAACTGTATTGGAGAAGATGGATGAATTA
CCCCAATTAACGTTGAGGTGAATAGGGGTCACAGAAGCATACCTCACACG
AACTCTATATCTGGTAGATGTGGATGGGAAGTGAATTGGAACTTCAATAT
ACCCTCTATTCTGAATGTTATTTCCACTGCTGTTGAGTCTAACGAGGTCT
CCACCAGTGAATCCTGGTCCTGAAATGACAGAACCGTTGAAGAGAAAGTT
TCCCTTCACTGCAGGGATTTGAGTAATACTATCGGATGCGATGATGTTGT
TGAACTCAGCACTACGGTGTATCCAAGAGAACATAGGAGCTCTGATGATG
CTCACGGAATTTTTGTTGAATCCGGAACGGAACATGGACACGTGGCTCAA
CCTGTGGGAGAATCCTTGCCTGGGTGGCACATTGTT

SEQ ID NO: 2

CCATGGACAACAACGTGCTCAACTCCGGCAGGACCACCATCTGCGATGCA
TACAACGTGGTGGCCCATGACCCGTTCTCCTTCGAGCATAAGTCCCTCGA
CACCATCCAGAAGGAGTGGATGGAGTGGAAGAGGACCGACCATTCCCTCT
ACGTGGCACCGGTGGTGGGCACCGTGTCCTCCTTCCTCCTCAAGAAGGTG
GGCTCCCTCATCGGCAAGAGGATTCTCTCCGAGCTCTGGGGCATCATCTT
CCCGTCCGGCAGCACCAACCTGATGCAGGACATCCTCAGGGAGACCGAGC
AGTTCCTCAACCAGAGGCTCAACACCGACACCCTCGCCCGTGTGAACGCC
GAGCTCATCGGCCTGCAGGCCAACATCAGGGAGTTCAACCAGCAGGTGGA
CAACTTCCTCAACCCGACCCAGAACCCGGTGCCGCTGTCGATCACCTCCT
CCGTGAACACCATGCAGCAGCTCTTCCTCAACAGGCTCCCGCAGTTCCAG
ATCCAGGGCTACCAGCTCCTCCTCCTCCCGCTCTTCGCCCAGGCCGCCAA
CATGCACCTCTCCTTCATCAGGGACGTGATCCTCAACGCCGACGAGTGGG
GCATCTCCGCCGCCACCCTCCGTACCTACAGGGACTACCTCAGGAACTAC
ACCAGGGACTACTCGAACTACTGCATCAACACCTACCAGACCGCCTTCAG
GGGCCTCAACACCAGGCTCCATGACATGCTCGAGTTCAGGACCTACATGT
TCCTCAACGTGTTCGAGTACGTGTCCATCTGGTCCCTCTTCAAGTACCAG
TCCCTCATGGTCTCCAGCGGCGCCAACCTCTACGCCTCCGGCTCCGGCCC
GCAGCAGACCCAGTCCTTCACCGCCCAGAACTGGCCGTTCCTCTACTCCC
TCTTCCAGGTGAACTCGAACTACATCCTCTCCGGCATCTCCGGCACCAGG
CTCTCCATCACCTTCCCGAACATCGGCGGCCTCCCGGGCTCCACCACCAC
CCATTCGCTGAACTCGGCCAGGGTGAACTACTCCGGCGGGCGTGTCCTCC
GGCCTCATCGGCGCCACCAACCTCAACCATAACTTCAACTGCTCCACCGT
GCTCCCGCCCTTAAGCACCCCGTTCGTGAGGTCCTGGCTCGACTCCGGCA
CCGACAGGGAGGGCGTGGCCACCTCCACCAACTGGCAGACCGAGTCCTTC
CAGACCACCCTCTCCCTCAGGTGCGGCGCCTTCTCCGCCAGGGGCAACTC
CAACTACTTCCCGGACTACTTCATCCGTAACATCTCCGGCGTGCCGCTCG
TGATCAGGAACGAGGACCTCACCAGGCCGCTCCATTACAACCAGATCAGG
AACATCGAGTCCCCGTCCGGCACCCCGGGCGGCGCCAGGGCCTACCTCGT
GTCCGTGCATAACAGGAAGAACAACATCTACGCCGCCAACGAGAACGGCA
CCATGATCCATCTCGCCCCGGAGGACTACACCGGCTTCACCATCTCCCCG
ATCCATGCCACCCAGGTGAACAACCAGACGCGTACCTTCATCTCCGAGAA
GTTCGGCAACCAGGGCGACTCCCTCAGGTTCGAGCAGTCCAACACCACCG
CCAGGTACACCCTCAGGGGCAACGGCAACTCCTACAACCTCTACCTCAGG
GTGTCATCGATCGGCAACTCCACCATCAGGGTGACCATCAACGGCAGGGT
GTACACCGTGTCCAACGTGAACACCACCACCAACAACGACGGCGTGAACG
ACAACGGCGCCAGGTTCTCCGACATCAACATCGCAACATCGTGGCCTCCG
ACAACACCAACGTCACTCTGGACATCAACGTCACCCTCAACTCCGGAACC
CCGTTCGACCTCATGAACATCATGTTCGTGCCGACCAACCTCCCGCCGCT
CTACTAAGGATCC

| | |
|---|---|
| ACAGAAGACCCTTCAATATC | SEQ ID NO: 3 |
| GTTACCGAGTGAAGATGTAA | SEQ ID NO: 4 |
| AGATTACCCCAGTTCCAGAT | SEQ ID NO: 5 |
| GTTCCCGAAGGACTTTCTAT | SEQ ID NO: 6 |

SEQ ID NO: 7

CCAGAGGCTGCACTTCTGGGTTACTCAGCAGTTGTATGGATGCATTCGTT
GATGTTTGGGTTGTGCCATGGAGATCTGCTAGAGTCAGCTTGTCAGCGTG
TCCTCTCCAATGAAATGAACTTCCTTATATAGAGGAAGGGTCTTGCGAAG
GATAGTGGGATTGTGCGTCATCCCTTACGTCAGTGGAGATATCACATCTA
TCTCCTTCGCTTTGAAGACGTGGTTGGAACGTCTTCTTTTTCCACGATGC
TCCTCGTGGGTGGGGGTCCATCTTTGGGACCACTGTCGGCAGAGGCATCT
TCAACGATGGCCTTTCCTTTATCGCAATGATGGCATTTGTAGGAGCCACC
TTCCTTTTCCACTATCTTCACAATAAAGTGACAGATAGCTGGGCAATGGA
ATCCGAGGAGGTTTCCGGATATTACCCTTTGTTGAAAAGTCTCATCGGAC
CATCACATCAATCCACTTGCTTTTGAAGACGTGGTTGGAACGTCTTCTTT
TTCCACGATGCTCCTCTTGGGTGGGGGGCCCTTCTTTGGGACACTGTCGG
AGAGG

SEQ ID NO: 8

GGGTTACTCAGCAGTTGTATGG

SEQ ID NO: 9

AAGTGGATTGATGTGATGGTC

### BRIEF DESCRIPTION OF THE FIGURE

FIG. 1 shows inserted cry1Ac & cry2A transgene and flanking sequences for cotton event CEMB02-451. This figure also shows amplicons and primers as described herein. Individual DNA sequences identified in FIG. 1 as numbers 1-7 are as follows:

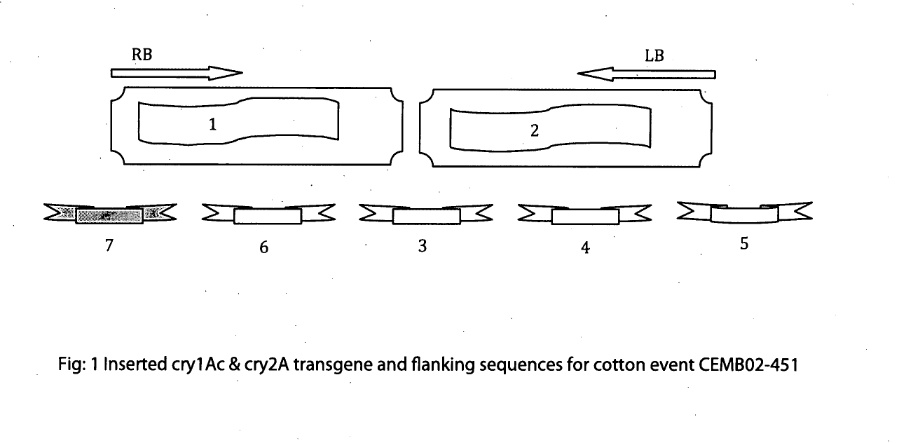

Reference number 1 is the DNA sequence for the Cry1Ac sequence and its border sequence (SEQ ID NO: 1).

Reference number 2 is the DNA sequence for the Cry2A Sequence and its border sequence (SEQ ID NO: 2).

Reference number 3 is the sequence of forward Cry1Ac primer to amplify a 565 bp fragment (SEQ ID NO: 3).

Reference number 4 is the sequence of reverse Cry1Ac primer to amplify a 565 bp fragment (SEQ ID NO: 4).

Reference number 5 is the sequence of forward Cry2A primer to amplify a 600 bp fragment (SEQ ID NO: 5).

Reference number 6 is the sequence of Reverse Cry2A primer to amplify 600 bp fragment (SEQ ID NO: 6).

Reference number 7 is the sequence of cotton event CEMB02-451 (SEQ ID NO: 7).

### BRIEF DESCRIPTION OF THE SEQUENCES

Sequence ID NO: 1 is the DNA sequence for the Cry1Ac sequence and its border sequence.

Sequence ID NO: 2 is the DNA sequence for the Cry2A Sequence and its border sequence.

Sequence ID NO: 3 is the sequence of forward Cry1Ac primer to amplify a 565 bp fragment

Sequence ID NO: 4 is the sequence of reverse Cry1Ac primer to amplify a 565 bp fragment

Sequence ID NO: 5 is the sequence of forward Cry2A primer to amplify a 600 bp fragment

Sequence ID NO: 6 is the sequence of Reverse Cry2A primer to amplify 600 bp fragment

Sequence ID NO: 7 is the sequence of cotton event CEMB02-451.

Sequence ID NO: 8 is the sequence of forward primer of event CEMB02-451 to amplify 451 bp fragment

Sequence ID NO: 9 is the sequence of Reverse primer of event CEMB02-451 to amplify 451 bp fragment

What is claimed is:

1. An insect resistant cotton plant, Gossypium hirsutum, seeds of said cotton plant having been deposited with the American Type Culture Collection.

2. An insect resistant cotton plant, containing into the plant's genome an insert DNA encoding Cry1Ac and Cry2A DNA having nucleotide sequences of SEQ ID NO: 1 and SEQ ID NO:2.

3. A progeny plant of the cotton plant of claim 1 or 2, wherein DNA of said progeny plant is capable of producing at least one amplicon of 565 bp and other of 600 bp using primers having the sequences of SEQ ID NO:3, SEQ ID NO:4, SEQ ID NO:5 and SEQ ID NO 6.

4. The said insect resistant cotton plant of claim 2, wherein the cotton plant is heterozygous for the Cry1Ac and Cry2A insert.

5. The said insect resistant cotton plant of claim 2, wherein the cotton plant is homozygous of the Cry1Ac and Cry2A insert.

6. A transgenic seed of the plant of claims 1, 2 or 3.

7. A transgenic seed of the plant of claim 2, wherein said seed comprises said nucleotide sequences.

8. A transgenic seed of the plant of claim 3, wherein said seed is capable of producing said amplicon.

9. A method of producing an insect resistant cotton plant comprising crossing the plant of claims 1, 2 or 3 with another cotton plant, and selecting insect resistant progeny by analyzing for at least one nucleotide sequence selected from the group consisting of SEQ ID NO:3, SEQ ID NO:4, SEQ ID NO:5 and SEQ ID NO:6.
